(19) United States
(12) Patent Application Publication
(10) Pub. No.: US 2025/0152524 A1
(43) Pub. Date: May 15, 2025

(54) TOPICAL COMPOSITIONS AND METHODS

(71) Applicant: VasoDynamics Limited, Stevenage (GB)

(72) Inventor: Ningfeng F LI, Stevenage (GB)

(73) Assignee: VasoDynamics Limited, Stevenage (GB)

(21) Appl. No.: 18/841,161

(22) PCT Filed: Mar. 4, 2022
(86) PCT No.: PCT/GB2022/050586

§ 371 Date: Aug. 23, 2024

(51) Int. Cl. A61K 31/137; A61K 9/00; A61K 47/10; A61P 39/00

(52) U.S. Cl. CPC A61K 31/137; A61K 9/0014; A61K 47/10; A61P 39/00

(57) ABSTRACT

The invention relates to pharmaceutical compositions comprising L-epinephrine or a pharmaceutically acceptable salt thereof; and a carrier vehicle comprising ethanol and water. The pharmaceutical composition has a molecular oxygen concentration of less than 5% wt/vol; and a pH of less than 3. The invention also relates to the use of the compositions in preventing chemotherapy or radiotherapy induced conditions. The invention also relates to methods of forming the pharmaceutical compositions.

### FIELD OF THE INVENTION

The present invention relates to pharmaceutical compositions comprising the alpha-adrenergic vasoconstrictor L-epinephrine and a carrier vehicle comprising ethanol and water. The invention also relates to the use of those pharmaceutical compositions for preventing chemotherapy and/or radiotherapy induced conditions in a patient, such as oral mucositis, changes to the speech and voice of a patient, dermatitis, alopecia, acute skin and mucosa inflammation, skin rash, skin ulceration, mucosa ulceration, decolouration, pigmentation, scaring and chronic fibrosis. The invention also relates to methods of forming the pharmaceutical compositions.

### BACKGROUND

Anti-cancer therapy frequently involves cytotoxic effects on the cancerous cells primarily, but also causes cytotoxic effects on non-malignant cells, tissues and organs.

Radiation-induced oral mucositis (RIOM) and chemotherapy-induced oral mucositis (CIOM) occur when irradiation and cytotoxic chemotherapy regimens destroy the mucosa dividing basal cells and consequently break down the cell layers lining the oral surface, leaving the mucosal tissue open to the development of ulceration and infection. It is the most common, debilitating, serious complication of cancer therapy, manifested as erythema, edema or ulcerative lesions. Oral mucositis is almost always painful, and it affects eating, sleeping, and speech, increases risk of infection due to open sores in the mucosa, and profoundly affects quality of life and cancer therapy continuity. Most importantly, it often leads to an interruption or discontinuation and change of the anti-cancer therapy regimes and consequently decreases the effectiveness of cancer control.

Similar basal cell damage and hair follicle stem cell damage induced by radiation exposure and/or cytotoxic chemotherapy can lead to acute dermatitis and alopecia and subsequent chronic ulceration or fibrotic changes.

Although skin and mucosa damage secondary to anti-cancer therapies may appear to be self-limiting and can partially or mostly heal after the cancer therapies are completed, it is particularly difficult to obtain optimal anti-cancer efficacy in patients who are physically and psychologically exhausted by the side-effects including the acute and chronic damages to normal skin and mucosa. In particular, oral mucositis secondary to anti-cancer therapies is considered as a dose limiting early effect since it leads to cancer therapy interruption, poor local tumour control, and changes in dose fractionation, which may prolong overall treatment time and compromise local control and overall survival. Radiation-induced oral mucositis side effects include oral pain in 69% of patients, dysphagia in 56% of patients, opioid use in 53% of patients, weight loss of 3-7 kg, feeding tube insertion and hospitalisation (ICU admission) in 15% of patients, and modification or interruption of treatment in 11-16% of patients (Maria O M, 2017).

Numerous efforts have been made to identify effective agents for the management of oral mucositis caused by chemotherapy in cancer patients. These interventions include aloe vera, amifostine, cryotherapy, granulocyte colonystimulating factor (G-CSF), intravenous glutamine, honey, keratinocyte growth factor, laser irradiation, polymyxin/tobramycin/amphotericin (PTA) antibiotic tablet/paste, sucralfate, natural and homeopathic agents, which offer some benefit in terms of the prevention or reduction of mucositis associated to cancer therapy. Among them, cryotherapy, palifermin and sucralfate showed statistically significant benefit in preventing or reducing the severity of mucositis induced by some types of chemotherapies. However, their use is not recommended routinely in patients treated for solid cancers such as head and neck cancers (De Sanctis V, 2016).

Currently, there is no single agent or management regimen that significantly improves the preventive effects of radiotherapy induced severe mucostitis to a clinically relevant and satisfactory standard, which indicates a significant unmet clinical need for many cancer patients (De Sanctis V, 2016; Maria, 2017; Chaveli-López B, 2016; Alvariño-Martín C, 2014).

The main therapeutic indications of epinephrine are for the treatment of low cardiac output, anaphylactic shock, acute bronchospasm, laryngotracheobronchitis and urticaria or angioedema. The actions of epinephrine are dose-dependent being mostly beta-stimulation with low concentrations, and alpha-stimulation with high concentrations increasing systemic vascular resistance and possibly decreasing the cardiac output in the latter. Epinephrine imparts a powerful bronchodilator action, most evident when bronchial muscle is contracted. Other than the intramuscular, intravenous, subcutaneous and intra-arterial routes of administration for the main clinical indications, human experience with topical epinephrine (i.e. inhaled, intraocular, topical) is extensive. Epinephrine may be added to solutions of some local anesthetics to decrease the rate of vascular absorption of the anesthetic. In addition, epinephrine is used locally to control superficial bleeding from arterioles and capillaries in the skin and mucous membranes of the eye, nose, mouth, throat or larynx, mainly during surgery. The drug is especially useful in dental surgery. In ophthalmology, topical epinephrine is used principally to reduce elevated IOP in the treatment of open-angle (chronic simple) glaucoma. Epinephrine may be applied topically to the nasal mucosa as a decongestant or used to temporarily relieve occasional symptoms of bronchial asthma and reversible bronchospasm (AHFS Drug Information 2009).

The PCT application WO 2006/138691 A1 describes the use of topical vasoconstrictors to protect cells during cancer chemotherapy and radiotherapy. We further developed a serial of L-epinephrine reformulation as oral topical mouth rinse and dermal topical solution/semi-solution to prevent radiation-induced acute oral mucositis, dermatitis and alopecia in cancer patients undergoing radiotherapy and/or chemoradiotherapy.

L-epinephrine and its salt forms such as epinephrine HCl, a nonselective adrenergic agonist, is an endogenous catecholamine secreted by the adrenal medulla in response to stress. It has marked effects on alpha-adrenergic and beta-adrenergic receptors, depending on the tissue distribution of the alpha- and beta-adrenergic receptors to which it binds. In general, alpha-1 and alpha-2-adrenergic receptors are present in the smooth muscle cells within blood vessel walls, particularly dermal blood vessels and are responsible for peripheral vasoconstrictions.

Early studies on serval animal models using topically applied vasoconstrictors such as epinephrine, norepinephrine and phenylephrine had shown successful outcome to prevent the mucositis, dermatitis and alopecia induced by ironing radiation (Soref, 2014; Soref, 2015; Fahl, 2016).

Another report also showed that on three xenograft tumour mice models, topical application of the adrenergic vasoconstrictors phenylephrine, epinephrine or norepinephrine had no interference to the curative radiation effect to cancerous tissues. This outcome was consistent with the historical observation that human tumour vasculature lacks the smooth muscle cells with adrenergic receptors that are required to enable a response to an adrenergic vasoconstrictor (Fahl, 2014; Graul-Conroy, 2016).

Furthermore, several clinical investigations have been carried out to preliminarily observe the safety and potential efficacy of topically applied norepinephrine and phenylephrine for the prevention/reduction of radiation or chemoradiation induced dermatitis, alopecia and oral mucositis (Cleary, 2017; Graul-Conroy, 2018). The preliminary clinical readout indicated a good safety profile of oral topical and dermal topical application of adrenergic vasoconstrictors on the studied cancer patients. However, norepinephrine or phenylephrine is half or one fifth fold less potent in comparison to epinephrine (Fahl, 2016). This leads to the attempt of further invention to develop the topical composition(s) using highly potent vasoconstrictor epinephrine, in particular, the active form of epinephrine namely L-epinephrine, as a drug substance, which can be applied oral topically and/or dermal topically with good pharmaceutical stability so to be utilised as pharmaceutical products for the prevention of oncology therapy induced mucositis, dermatitis and alopecia in cancer patients.

### SUMMARY OF INVENTION

The present invention discloses pharmaceutical compositions comprising the vasoconstrictor L-epinephrine, as well as methods in which these pharmaceutical compositions are applied topically to oral mucosa, skin or scalp in experimentally-determined concentrations and times to prevent or suppress side effects that are commonly encountered in the course of cancer therapies.

In a first aspect of the invention there is provided a pharmaceutical composition comprising 0.9 mg/ml to 15 mg/ml L-epinephrine or a pharmaceutically acceptable salt thereof. The composition also comprises a carrier vehicle comprising ethanol and water. The pharmaceutical composition has a molecular oxygen concentration of less than 5% wt/vol, and a pH of less than 3.

It has been surprisingly and advantageously found that this pharmaceutical composition is particularly effective at penetrating the oral mucosa to deliver the adrenergic vasoconstrictor L-epinephrine to the submucosal vasculature of the oral cavity to induce transient vasoconstriction and hypoxia at the treated site. In subsequent exposure to radiotherapy, the lack of oxygen at the treated area leads to any ROS-modified DNA nucleotides that have been produced to decay (in milliseconds) back to their original basal state with no discernible radiation toxicity.

The constriction of blood vessels due to the pharmaceutical compositions of the invention is also capable of protecting healthy cells at the treated area from any systemic chemotherapy agents. This is due to the reduced amount of blood supply with cytotoxic drugs and inflammation cytokines/chemokines at the vasoconstricted site, which reduces the amount of exposure to these damaging agents.

Consequently, death of normal, healthy cells of the oral mucosa due to radiotherapy and/or chemotherapy is mitigated by the pharmaceutical compositions of the present invention.

Furthermore, the pharmaceutical formulation also exhibits a high degree of stability compared to formulations of the prior art. Therefore, this represents an improved and promising treatment for radiotherapy-induced or chemotherapy-induced conditions affecting the oral mucosa.

Accordingly, the second aspect of the invention provides a pharmaceutical composition according to the first aspect for use in the prevention of chemotherapy-induced and/or radiotherapy-induced oral mucositis and/or changes to speech and voice in a patient. The pharmaceutical composition is for orotopical administration to an oral surface or oropharyngeal cavity surface of the patient.

In a third aspect, the invention provides a pharmaceutical composition comprising 0.92 mg/ml to 36.6 mg/ml L-epinephrine or a pharmaceutically acceptable salt thereof. The composition also comprises a carrier vehicle comprising ethanol and water. The pharmaceutical composition has a molecular oxygen concentration of less than 5% wt/vol; and has a pH of less than 3.

The applicant has found that this pharmaceutical composition is particularly suitable for transcutaneous delivery of L-epinephrine to the subcutaneous vasculature of the skin or scalp to induce transient vasoconstriction and hypoxia at the treated site. If the treated area is subsequently exposed to radiotherapy, the lack of oxygen due to the effects of the vasoconstrictor results in the mitigation of the death of normal, healthy squamous skin cells.

The constriction of blood vessels also prevents cell damage from chemotherapy agents by reducing the amount of blood carrying these agents in the vicinity of cells at the treated site.

Consequently, death of normal, healthy cells at the skin epidermis or hair follicle due to radiotherapy and/or chemotherapy is mitigated.

Furthermore, the pharmaceutical formulation also exhibits a high degree of stability compared to formulations of the prior art. Therefore, this represents an improved and promising treatment for radiotherapy-induced or chemotherapy-induced conditions affecting the squamous skin or scalp.

Therefore, in a fourth aspect, the invention provides a pharmaceutical composition according to the third aspect for use in the prevention of radiotherapy induced conditions selected from the group comprising dermatitis, alopecia, acute skin and mucosa inflammation, skin rash, skin ulceration, mucosa ulceration, decolouration, pigmentation, scaring and chronic fibrosis. The composition is for topical administration to the squamous skin and/or the scalp of a patient.

The fifth aspect of the invention provides a method of forming a pharmaceutical composition. The method comprises the steps of:
a) adding water to a vessel;
b) purging the water in the vessel with nitrogen gas until the dissolved molecular oxygen content reaches 5% wt/vol or less;
c) adding ethanol followed by at least one antioxidant to the vessel to form a composition;
d) adding at least one adrenergic vasoconstrictor or a pharmaceutically acceptable salt thereof to the composition; and
e) adjusting the pH of the composition to less than 3.

The composition formed by this method has a molecular oxygen concentration of less than 5% wt/vol.

The method leads to the formation of highly stable pharmaceutical compositions which are capable of retaining their therapeutic efficacy following long-term storage.

### BRIEF DESCRIPTION OF THE DRAWINGS

FIG. 1 shows a schematic of a mechanism of action of topically applied adrenergic vasoconstrictors to protect normal skin/mucosa from radiation and chemotherapy damage, which thereby enhances anticancer therapy.

FIG. 2 shows the stepwise HCl titration of L-epinephrine base dissolved in an orotopical delivery vehicle to achieve a final formulation pH of 2.50.

FIG. 3 shows the HPLC determination of L-epinephrine and adrenochrome peak areas in samples of “L-epi-Base” (pH 2.50) as well as “L-epi-HCl” (pH 5.05) stored at 40° C. in the dark for 10 days.

FIG. 4 (from Kerr et al, J Dental Res, 1991) outlines the typical internal surface area of the human oral cavity.

FIG. 5 illustrates lip blanching tests on four healthy volunteers with four concentrations of L-epinephrine in an orotopical solution. The lip blanching photos demonstrate blanching after applying 5.5 mg/ml L-epinephrine solution on the lower lip. The symbols, and their meanings are as follows: −no blanch response; +slight blanch response; ++moderate blanch response; +++significant blanch response.

FIG. 6 (from Fahl W E., Arch Dermatol Res. 2016 December) shows the relative potency with which three adrenergic vasoconstrictors epinephrine, norepinephrine and phenylephrine, suppressed radiation-induced dermatitis in the 13 days following irradiation with 17.5 Gy of a skin patch on the backs of shaved rats.

FIG. 7 illustrates a skin blanching test on a healthy volunteer's forearm with dermal topical application of a pharmaceutical composition containing 3 mg/ml L-epinephrine and ethanol. The treated area of the skin shows visible blanching, and the blanching appears significant around the hair roots area (arrows), especially starting from 14 minutes after the dermal topical application.

FIG. 8 shows that the pharmaceutical formulation including a carrier vehicle comprising ethanol and water, and L-epinephrine, which is deoxidised to have a molecular oxygen concentration of less than 5% wt/vol, has a pH above 6. An excess amount of HCl is added into the formulation to achieve a final pH lower than 3, preferably pH at about 2.3.

The additional volume of HCl increases following a linear correlation to the concentration of L-epinephrine. The correlation can be described as the equation below:

$$Y = 5 \text{.6297} * X - 5.3 \; 044 , correlation ratio R 2 = 0 \text{.9916}$$

$$X \text{:} L - epinephrine concentration in mg / ml ;$$

$$Y \text{:} Additional volume of 2 N HCl in the pharmaceutical composition .$$

The correlation is calculated based on the measurements collected during the production of the carrier vehicle formulation without L-epinephrine, 0.92 mg/ml L-epinephrine and 5.5 mg/ml L-epinephrine formulation solutions

FIG. 9 shows the blanch responses in a human subject's forearm 30 minutes after dermal topical application of a pharmaceutical composition comprising L-epinephrine, a carrier vehicle comprising ethanol and water which is deoxidised to have a molecular oxygen concentration of less than 5% wt/vol and a pH adjusted to 2.33.

Forearm skin blanching tests (from left to right):
1. L-epi 3.6 mg/ml formulation with pH 2.33, ethanol and deoxidised; blanching after 30 minutes skin application.
2. L-epi 3.6 mg/ml formulation with pH 2.33 and deoxidised without ethanol; no blanching.
3. L-epi 3.6 mg/ml formulation with pH 2.33 without antioxidant; no blanching.
4. L-epi 3.6 mg/ml formulation with pH 5.5 and deoxidised; no blanching.
5. L-epi 3.6 mg/ml formulation with pH 2.33 and deoxidised with daylight exposure for 24 hours; blanching after 30 minutes skin application.

The absence of blanching responses from the solution without ethanol, or without deoxidant, or without pH adjusted to lower than 3, further confirms the essential contributions to the bioavailability and stability of L-epinephrine from the combination of a carrier vehicle comprising ethanol and water which is deoxidised to have an oxygen content of less than 5% wt/vol and pH lower than 3.

FIG. 10 is a photograph of a pharmaceutical composition comprising L-epinephrine at 5 mM (0.92 mg/ml) with a molecular oxygen concentration of 20% wt/vol when subjected to temperatures of 40° C. and 60° C. (Example 9). Under the 60° C. high temperature stress condition, the sample colour turned from clear and colourless to light brown.

### DETAILED DESCRIPTION

In a first aspect, the invention provides a pharmaceutical composition comprising 0.9 mg/ml to 36.6 mg/ml L-epinephrine or a pharmaceutically acceptable salt thereof. Additionally, the composition comprises a carrier vehicle comprising ethanol and water. The pharmaceutical composition has a molecular oxygen concentration of less than 5% wt/vol. The final pharmaceutical composition has a pH of less than 3.

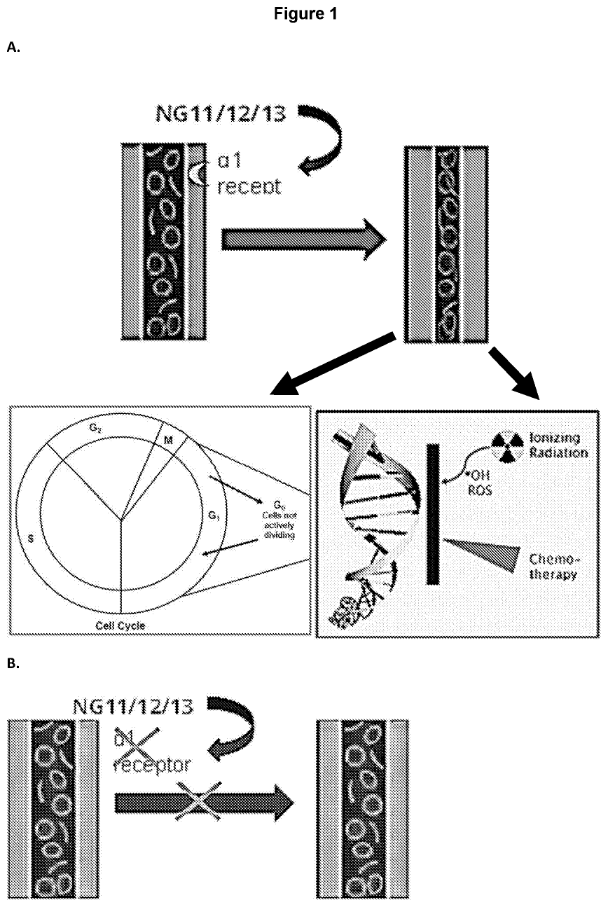

The pharmaceutical composition has been developed to protect the normal epithelial cells of the oral mucosa in a patient from the toxic effects of therapy with radiation or chemotherapeutic agents, commonly used in cancer treatment. This can be understood with reference to FIG. 1.

FIG. 1 shows a schematic of a mechanism of action of topically applied adrenergic vasoconstrictors to protect normal skin/mucosa from radiation and chemotherapy damage, which thereby enhances anticancer therapy.

Part A of FIG. 1 illustrates the mechanism with respect to normal skin/mucosa blood vessels. A topically applied vasoconstrictor induces adrenergic alpha receptor mediated successful vasoconstriction as there are intact smooth muscles within the wall of the peripheral vessels in the normal skin/mucosa. Transient induction of vasoconstriction produces transient hypoxia conditions around the basal cells, temporary switching the cells to GO cell cycle—this ‘hides’ the radiation-vulnerable basal cells from radiation damage. After the radiation exposure, the transient vasoconstriction effect diminishes, the undamaged basal cells re-enter the divisional cycle post treatment and enhances an accelerated healing of skin/mucosa structure. Meanwhile, reduced blood supply minimises the production of Reactive Oxygen Species (ROS) and consequently reduces DNA damage from ROS. About 70% of cytotoxic effects from ionising radiation and chemotherapy are due to ROS-mediated DNA damage. Hence, minimising the production of ROS largely contributes to the protective effects of topically applied vasoconstrictors on the basal cells against radiation damage and chemotherapy damage. Furthermore, reduced blood supply can also reduce the inflammatory cytokines and chemokines, subsequently reducing inflammation development and symptoms.

Part B of FIG. 1 shows the mechanism with respect to cancerous tissue blood vessels. These cancerous tissue blood vessels are mostly immature and lack the alpha adrenergic receptorcontaining smooth muscles in the vessel wall, hence topically applied adrenergic vasoconstrictors do not induce vasoconstriction and hypoxia condition in cancerous tissues. Thus, the topically applied vasoconstrictors do not protect cancerous tissues from radiotherapy and chemotherapy damage.

The claimed concentration of L-epinephrine is sufficient for transiently constricting submucosal or subcutaneous blood vessels to reduce the amount of oxygenated blood arriving at stem cells within the mucosa, epidermis or hair follicle when topically applied to the oral mucosa, skin or scalp of a patient.

In subsequent exposure to radiotherapy, the lack of oxygen at the treated area leads to any ROS-modified DNA nucleotides that have been produced to decay (in milliseconds) back to their original basal state with no discernible radiation toxicity.

The constriction of blood vessels due to the pharmaceutical compositions of the invention is also capable of protecting healthy cells at the treated area from any systemic chemotherapy agents due to the reduced amount of oxygenated blood at the treated area. Consequently, death of normal, healthy cells in the vicinity of the treated area is mitigated.

The pure, L-epinephrine enantiomer is twice as potent as the racemic +/−epinephrine HCl that is used in many injectable epinephrine products, as pharmacologic dilution with the inactive R-enantiomer is eliminated (Nutman et al., Crit Care Med 1994). Existing art that describes formulation of the racemic +/−epinephrine HCl salt or the (−) epinephrine bitartrate salt provides no guidance on how to formulate an aromatic free base form of L-epinephrine in a water plus alcohol carrier which is required for transmucosal and transcutaneous delivery for orotopical and topical administration, and in which the free base is chemically insoluble. Therefore, the present inventors have devised specific conditions in which the pure L-enantiomer can be effectively used for delivery across oral mucosa, and across squamous skin or into scalp hair follicles.

The L-epinephrine contained within the pharmaceutical compositions of the invention may be present as a pharmaceutically acceptable salt. A “pharmaceutically acceptable salt” is intended to mean a salt of a free acid or base of a compound that is non-toxic, biologically tolerable, or otherwise biologically suitable for administration to a subject. Such pharmaceutically acceptable salts are known to those skilled in the art.

Examples of suitable pharmaceutically acceptable salts are those that are pharmacologically effective and suitable for contact with the tissues of subjects without undue toxicity, irritation, or allergic response. A compound of the invention, may possess a sufficiently acidic group, a sufficiently basic group, or both types of functional groups, and accordingly react with a number of inorganic or organic bases, and inorganic and organic acids, to form a pharmaceutically acceptable salt.

Pharmaceutically acceptable acid addition salts can be formed with inorganic acids and organic acids, and pharmaceutically acceptable base addition salts can be formed with inorganic and organic bases.

Examples of pharmaceutically acceptable salts particularly include sulfates, sulfites, phosphates, chlorides, hydrochlorides, bromides, iodides, acetates, propionates, acrylates, formates, benzoates, sulfonates, citrates, lactates, glycolates, tartrates and bitartrates.

Particularly preferred salts of L-epinephrine include L-epinephrine hydrochloride, and L-epinephrine bitartrate.

The inventors have found that ensuring that the dissolved oxygen content in the pharmaceutical compositions is less than 5% wt/vol significantly improves the stability of the active pharmaceutical ingredient L-epinephrine, and enables it to retain stability for at least 3 months. They have also found that ensuring that stability is improved when the aerosol phase of the final product also has an oxygen content below 5% wt/vol in the final container/vial. This provides an improvement over known compositions with an oxygen concentration of more than 5% wt/vol which result in the active pharmaceutical ingredient L-epinephrine becoming easily oxidized and degraded, which in turn diminishes its therapeutic efficacy.

Methods of measuring the oxygen content of a solution are known to the person skilled in the art. In the methods described in the Examples herein, an Oxysense 325i industrial oxygen meter comprising a probe is used.

As demonstrated by the Examples herein, compositions comprising L-epinephrine and a carrier vehicle comprising water and ethanol do not inherently have less than 5% wt/vol oxygen unless the oxygen is actively controlled and reduced. This can be achieved during the manufacturing process by adding an antioxidant, such as sodium metabisulfite, to neutralise the oxygen in combination with nitrogen purging.

Ensuing that the carrier solution has a pH of less than 3 advantageously improves the dissolution and stabilisation of L-epinephrine free base. As a result, the pharmaceutical composition according to the invention comprises more dissolved, bioavailable L-epinephrine drug molecules to induce vasoconstriction, resulting in improved efficacy.

The pharmaceutical composition may comprise hydrochloric acid. This component may be added to the pharmaceutical composition to reach the pH required by claim 1. Hydrochloric acid may be the only further acidic compound added to lower the pH. Alternatively, hydrochloric acid may be added in combination with other acidic compounds to lower the pH of the pharmaceutical composition.

The additional volume of hydrochloric acid in the composition may follow a linear correlation relative to the concentration of L-epinephrine. In some embodiments, the linear correlation equation can be described as:

$$Y = 5.6297 * X - 5.3 \; 044 , correlation ratio R 2 = 0 \text{.9916}$$

$$X \text{:} L - epinephrine concentration in mg / ml ;$$

$$Y \text{:} Additional volume of 2 N HCl in pharmaceutical composition according to claim 1 .$$

Hydrochloric acid lowers the pH of the pharmaceutical composition, which allows for effective solubilising of the L-epinephrine, which in turn enables its effective application to patients. The lowered pH also improves the long-term storage stability of the pharmaceutical composition, which results in improved commercial products with longer shelf lives.

The carrier vehicle comprising ethanol and water enables the pharmaceutical composition to be suitable for transcutaneous delivery of the L-epinephrine to the subcutaneous vasculature of the skin or scalp, or orotpically delivered to the submucosal vasculature of the oral cavity.

In one embodiment in accordance with the first aspect of the invention, the carrier vehicle further comprises glycerol and/or propylene glycol, preferably both glycerol and propylene glycol. This carrier vehicle is particularly suitable for transcutaneous delivery of L-epinephrine for topical application of the pharmaceutical composition on the skin or scalp.

Preferably, the carrier vehicle of this embodiment comprises from 2% to 10% vol/vol ethanol; from 2% to 10% vol/vol glycerol; from 4% to 12% vol/vol propylene glycol; and from 70% to 90% vol/vol water. In one embodiment according to the first aspect of the invention, the carrier vehicle comprises ethanol:glycerol:propylene glycol:water in a 6:6:8:80 vol:vol ratio. It is also envisaged that the carrier vehicle according to the invention may exclude (i.e. not include) ethanol:glycerol:propylene glycol:water in the particular vol:vol ratio 6:8:8:80 vol:vol.

The pharmaceutical composition according to the first aspect of the invention comprises L-epinephrine or a pharmaceutically acceptable salt thereof in an amount of from 0.9 mg/ml to 15 mg/ml. Preferably, the L-epinephrine or a pharmaceutically acceptable salt thereof is present in the range of 0.9 mg/ml to 11 mg/ml, 0.9 mg/ml to 9 mg/ml, 1.8 mg/ml to 9 mg/ml, 1.8 mg/ml to 7.5 mg/ml, 2.7 mg/ml to 7.5 mg/ml, most preferably 2.7 mg/ml to 5.5 mg/ml. Alternatively, L-epinephrine may be contained in an amount of 0.9 mg/ml to 5.5 mg/ml.

The concentration of L-epinephrine in units of mM, which correspond to the above values in mg/mL, may be from 5 mM to 80 mM, preferably 5 mM to 60 mM, 5 mM to 50 mM, 10 mM to 50 mM, 10 mM to 40 mM, 15 mM to 40 mM, most preferably 15 mM to 30 mM. Alternatively, L-epinephrine may be contained in an amount of 5 mM to 30 mM.

The pharmaceutical composition according to the invention has a molecular oxygen content of less than 5% wt/vol. Preferably, the pharmaceutical composition has a molecular oxygen concentration of less than 4.5% wt/vol, preferably less than 4.0% wt/vol, preferably less than 3.5% wt/vol, preferably less than 3.0% wt/vol, and most preferably less than 2.5% wt/vol.

In addition to L-epinephrine, the pharmaceutical composition may further comprise additional vasoconstrictors, such as D-epinephrine, DL-epinephrine and/or pharmaceutically acceptable salts thereof.

The pharmaceutical composition may also further comprises one or more antioxidants, preferably in an amount of from 0.01 to 0.5% wt/vol. The antioxidant is capable of inhibiting oxidation that can produce ROS and chain reactions that may damage cells.

Preferably the antioxidant is selected from the group comprising a bisulfite salt such as sodium metabisulfite, citric acid, vitamin C, vitamin E, L-cysteamine or combinations thereof. Most preferably, the antioxidant is sodium metabisulfite, citric acid or combinations thereof.

The pharmaceutical composition of the invention may comprise one or more penetration enhancers. Examples of suitable penetration enhancers include isopropyl myristate (supplied by Tegosoft M) or medium-chain monoglycerides.

The pharmaceutical composition according to the invention may comprise one or more keratolytic ingredients. An example of a suitable keratolytic ingredient is lactic acid.

The pharmaceutical composition may comprise one or more sweeteners, preferably in an amount of from 0.01 to 5% wt/vol. Alternatively, or in addition to the sweeteners, the pharmaceutical composition may comprises one or more flavouring agents, preferably in an amount of from 0.05 to 2% wt/vol. The inclusion of sweeteners and/or flavouring agents in the pharmaceutical compositions improves their palatability to patients and enables its prolonged topical application to the oral cavity. The composition comprises a sucralose sweetener and/or a cherry flavouring agent.

It is envisaged that the invention may explicitly exclude (i.e. does not include) pharmaceutical compositions comprising the following features in combination:
0.01 to 0.5% wt:vol of one or more antioxidants;
0.01 to 5% wt:vol of one or more sweeteners; and
0.05 to 2% wt:vol of one or more flavouring agents.

It is envisaged that the invention may also explicitly exclude (i.e. not include) pharmaceutical compositions comprising a carrier vehicle comprising ethanol: glycerol:propylene glycol:water in the specific ratio 6:6:8:80 vol:vol; in combination with 0.01 to 5% wt:vol of one or more sweeteners; and/or 0.05 to 2% wt:vol of one or more flavouring agents.

The composition may be formulated in numerous ways. Preferably, the composition is formulated in a way that enables simple and effective orotopical application to the oral mucosa of a patient. Preferably, such compositions are formulated as a mouth rinse, gel, balm, oil, paste or aerosol.

According to a second aspect, the invention provides a pharmaceutical composition according to the first aspect for use in the prevention of chemotherapy-induced and/or radiotherapy induced oral mucositis in a patient and/or changes to speech and voice in a patient. The composition is for orotopical administration to an oral surface or oropharyngeal cavity surface of the patient.

The method of use of the orotopical formulation developed by the applicant ensures that sufficient drug molecule, drug formulation volume, and contact time of the drug formulation with the oral mucosa to enable rapid, efficient delivery of sufficient L-epinephrine to submucosal blood vessels within the time constraints of clinical practice (minutes). This results in the rapid onset and sufficient duration of vasoconstriction to protect against a short-lived (seconds-minutes) radiotherapy insult, or through recurrent applications against a longer chemotherapy insult.

L-epinephrine may be applied orotopically to oral mucosa in a 1-3 minute, preferably in 1.5-2.5 minute “swish and spit” administration (i.e. mouth rinse). This provides several advantages over the existing oral cryotherapy approach, including:
i) there is none of the discomfort associated with holding a mouth full of ice;
ii) it can be used in head and neck radiotherapy patients where ice cannot;
iii) the adrenergic agonist-induced vasoconstrictor effect can be controlled in a predictable manner through standard dose and application time strategies, i.e., a single vasoconstrictor application whose transient hypoxic effect dissipates quickly before a daily radiotherapy session, or, multiple applications over several hours to sustain the exclusion of blood-borne chemotherapy from the oral mucosa through 1-2 plasma half-lives of the systemic chemotherapy drug(s);
iv) protection of the oral cavity and tongue surface epithelia that contact the orotopically administered vasoconstrictor may also confer protection to taste buds.

The pharmaceutical composition for use according to the second aspect is preferably for administration in a dose suitable to provide from 0.03-3 milligram L-epinephrine/cm² on the treated oral surface or oropharyngeal cavity surface of the patient. The applicant has determined that the specific formulation concentration range of 0.9 mg/ml-7.5 mg/ml L-epinephrine is associated with an orotopical dose range of 0.03 mg-3 mg L-epinephrine per cm² of mucosal surface.

Preferably, the pharmaceutical composition for use is for administration to the oral surface or oropharyngeal cavity surface of a patient undergoing radiotherapy and/or chemoradiotherapy. It may be administered no more than 50 minutes prior to a first and each subsequent fractional radiation exposure to the treated oral or oropharyngeal cavity surface.

Preferably, it is administered no more than 45 minutes, preferably no more than 40 minutes, preferably no more than 35 minutes, preferably no more than 30 minutes prior to radiation exposure.

In one embodiment, the L-epinephrine pharmaceutical composition may be delivered orotopically once for a 30 seconds to 2 minutes period of mouth rinse to confer protection to the oral mucosa from a daily dose of 0.5-5 Gy of head and neck area radiotherapy.

In a third aspect, the invention provides a pharmaceutical composition comprising 0.92 mg/ml to 36.6 mg/ml L-epinephrine or pharmaceutically acceptable salt thereof. The composition comprises a carrier vehicle comprising ethanol and water. The pharmaceutical composition has a molecular oxygen concentration of less than 5% wt/vol; and a the pH of the pharmaceutical composition is less than 3.

This pharmaceutical composition is particularly suitable for protecting the normal epidermal stem cells of squamous skin as well as the specialized stem cells of the scalp epidermis and hair follicles against the toxic effects of therapy with radiation or chemotherapeutic agents.

Preferably, the pharmaceutical composition according to the third aspect comprises hydrochloric acid. This component may be added to the pharmaceutical composition to reach the pH required by claim 13. Hydrochloric acid may be the only further acidic compound added to lower the pH. Alternatively, hydrochloric acid may be added in combination with other acidic compounds to lower the pH of the pharmaceutical composition.

The additional volume of hydrochloric acid in the composition may follow a linear correlation relative to the concentration of L-epinephrine. In some embodiments, the linear correlation equation can be described as:

$$Y = 5.6297 * X - 5.3 \; 044 , correlation ratio R 2 = 0 \text{.9916}$$

$$X \text{:} L - epinephrine concentration in mg / ml ;$$

$$Y \text{:} Additional volume of 2 N HCl in pharmaceutical composition according to claim 1 .$$

Hydrochloric acid lowers the pH of the pharmaceutical composition, which allows for effective solubilising of the L-epinephrine, which in turn enables its effective application to patients. The lowered pH also improves the long-term storage stability of the pharmaceutical composition, which results in improved commercial products with longer shelf lives.

The carrier vehicle of the pharmaceutical composition preferably comprises from 60% to 80% vol/vol ethanol; and from 20% to 40% vol/vol water. In an embodiment, the ratio of ethanol:water in the carrier vehicle is 70:30 vol:vol. The carrier vehicle is suitable for transcutaneous delivery of the L-epinephrine to the subcutaneous vasculature of the skin or scalp. The invention may explicitly exclude pharmaceutical compositions with a carrier vehicle comprising ethanol:water in the specific ratio of 70:30 vol:vol.

Preferably, one or more antioxidants are contained within the pharmaceutical composition. They are preferably contained in an amount of from 0.01 to 0.5% wt/vol.

The pharmaceutical composition according to the third aspect may be formulated in a number of ways suitable for topical application to the skin or scalp of a patient. Preferably, the composition is formulated as a gel, shampoo, balm, oil, paste or cream.

According to a fourth aspect, the invention provides a pharmaceutical composition according to the third aspect of the invention for use in the prevention of radiotherapy induced conditions. The conditions are selected from the group comprising dermatitis, alopecia, acute skin inflammation, rash, ulceration, mucosa ulceration, decolouration, pigmentation, scaring and chronic fibrosis. The pharmaceutical composition is for topical administration to the squamous skin and/or the scalp of a patient.

The method of use of the topical formulation developed by the applicant ensures that sufficient drug molecule, formulation volume, and contact time of the drug formulation with the squamous skin or scalp skin to enable rapid, efficient delivery of sufficient L-epinephrine to subcutaneous blood vessels within the time constraints of clinical practice (minutes). This results in the rapid onset and sufficient duration of vasoconstriction to protect against a short-lived (seconds-minutes) radiotherapy insult, or through recurrent topical applications to the scalp against a longer chemotherapy insult. This is based upon the scalp blanch times/unit L-epinephrine doses determined by the inventors.

In one embodiment, the pharmaceutical composition is for administration in a dose suitable to provide from 0.03 mg-3 mg L-epinephrine/cm² on the treated squamous skin surface or scalp surface of a patient. The applicant has determined that the specific formulation concentration range of 0.9 mg/ml-15 mg/ml L-epinephrine is associated with a topical dose range of 0.03 mg-3 mg L-epinephrine per cm² of skin or scalp surface.

Preferably, the composition is for administration to the squamous skin surface or scalp surface of a patient undergoing radiotherapy and/or chemoradiotherapy. Preferably, the composition is for administration between 5 to 50 minutes prior to a first and each subsequent fractional radiation exposure to the treated squamous skin or scalp surface.

Preferably, the pharmaceutical composition is administered no more than 50 minutes, preferably no more than 45 minutes, preferably no more than 40 minutes, preferably no more than 35 minutes, preferably no more than 30 minutes prior to radiation exposure.

In one embodiment, the L-epinephrine pharmaceutical composition may be delivered topically directly to the scalp surface of a patient with or without an applicator no more than 40 minutes before radiation exposure to skin and scalp, to confer protection of the scalp hair follicle stem cells from fractional radiation during the course of radiotherapy treatment of a patient.

In another embodiment, the L-epinephrine pharmaceutical composition may be administered topically to the scalp, such as once every two hours, preferably for 4-6 hours, to enable exclusion of blood-borne chemotherapy from the hair follicle stem cells for an extended period and thus reduce the unwanted killing of the hair follicle stem cells as a side effect of the systemic chemotherapy use. Exclusion of blood-borne chemotherapy for 4-6 hours from scalp hair follicle stem cells may be sufficient to enable anywhere from one to several clearance half-lives for most cancer chemotherapy drugs from the patients' blood, thereby reducing the dose of chemotherapy to the hair follicle stem cells to a level that enables sufficient stem cell survival and sustained propagation of the hair shaft growth.

A fifth aspect of the invention provides a method of forming a pharmaceutical composition. The method comprises the steps of:
a) adding water to a vessel;
b) purging the water in the vessel with nitrogen gas until the dissolved molecular oxygen content reaches 5% wt/vol or less;
c) adding ethanol followed by at least one antioxidant to the vessel to form a composition;
d) adding at least one alpha-adrenergic vasoconstrictor to the composition; and
e) adjusting the pH of the composition to less than 3.

The composition has a molecular oxygen concentration of less than 5% wt/vol.

The antioxidant may be selected from the group comprising citric acid, vitamin C, vitamin E, L-cysteamine, a bisulfite salt, or combinations thereof. Preferably, the antioxidant is selected from citric acid or sodium metabisulfite or combinations thereof.

The alpha-adrenergic vasoconstrictor may be selected from L-epinephrine, D-epinephrine, norepinephrine, phenylephrine or mixtures thereof. Most preferably, the alpha-adrenergic vasoconstrictor is L-epinephrine.

The alpha-adrenergic vasoconstrictor is charged to the vessel after the components in step c) are added. The present inventors found that this particular sequence resulted in improved antioxidation effects and stability.

In step c) of the method, at least one stabiliser may be added to the composition. Preferably, the stabiliser is glycerol, propylene glycol, or mixtures thereof.

At least one flavouring agent may be added to the composition in step c). Preferably, the flavouring agent is sucralose, citric acid or mixtures thereof.

Components such as citric acid may simultaneously function as both an antioxidant and a flavouring agent.

In step e), the pH of the composition is preferably adjusted to less than 2.5. The lowering of the pH in step e) may be achieved by adding hydrogen chloride.

The method according to the fifth aspect may further comprises the step of:
f) filtering the composition with filter pore sizes of 0.3-0.5 μm and then 0.18-0.28 μm.

In one embodiment, the composition is filtered with a filter pore size of 0.45 μm followed by 0.22 μm.

The purpose of the filtration step is to homogenise and sterilise the composition. This is an important step before the composition is added to a container such as a vial or other package, which is suitable for storing the composition before use. The filtration step ensures that unwanted particles such as bacteria and fungi are removed from the composition, in addition to any undissolved large particles.

Prior to step a), the method may involve the step of purging the air in the vessel with nitrogen until the oxygen concentration of the air in the vessel is less than 5% wt/vol.

### EXAMPLES

### Example 1

Experiments were conducted to determine conditions under which the L-epinephrine free base, which is the only chemical form of L-epinephrine available in the cGMP form acceptable for pharmaceutical formulation, could be solubilized in the water:alcohol-based delivery vehicle that we had previously determined to be best suited for delivery of catecholamine vasoconstrictors to mammalian oral mucosa.

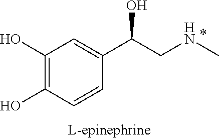

The applicant reasoned that step-wise additive titration with HCl would result in the formation of an HCl salt of the L-epinephrine amine group (* in schematic), and this would i) enable dissolution of the otherwise insoluble L-epinephrine base in the water:alcohol vehicle to achieve the required L-epinephrine concentration for topical delivery, and ii) would provide stability to the dissolved L-epinephrine to enable us to formulate and then hold in storage for a minimum of 18 months at room temperature or below.

In the Experiment 1 titration, solid L-epinephrine free base powder was added to the water:alcohol oral delivery vehicle (** in Table 1), 5 N HCL was titrated in with stirring until a clear solution was achieved, and a 1.0 ml sample was applied topically to a human test subject's lips to observe the time to induction of the blanch response in the oral mucosa tissue. The results are summarized in Table 1. At pH 4.95, the solution was clear and colourless, and it took ˜20 min to see a discernible lip blanch response. 18 hours later, the clear solution had developed a pink colour (consistent with adrenochrome formation, an oxidative epinephrine metabolite) and flocculant, re-precipitated material had appeared on the surface of the liquid.

TABLE 1

| | | | Time (min) | |
|---|---|---|---|---|
| | | | to Discernible | Lip |
| | | Appearance | Lip Blanch | Blanch |
| | | of Beaker | (after applying | Re- |
| Beaker Contents | pH | Contents | 1.0 ml to lips) | sponse |
| 1. Orotopical delivery | 4.09 | Clear | | |
| vehicle** | | | | |
| 2. Added 20 mM L- | 7.32 | Turbid, | | |
| epinephrine free base | | chalky | | |
| 3. Added 77 ul of 5N | 4.95 | Clear | 18-24 minutes | Dis- |
| HCl in 10 ul aliquots | | | | cernible |
| 4. Same as #3, +18 | 4.98 | Clear, light | | |
| hr at room temp. | | pink color, | | |
| | | flocculant | | |
| | | material on | | |
| | | liquid surface | | |

**Orotopical delivery vehicle consisted of: Nitrogen gas-purged ethanol:glycerol:propylene glycol:water (6:6:8:80 vol:vol), 0.025% sucralose sweetener (wt:vol), 0.2% sodium metabisulfite, 0.1% citric acid.

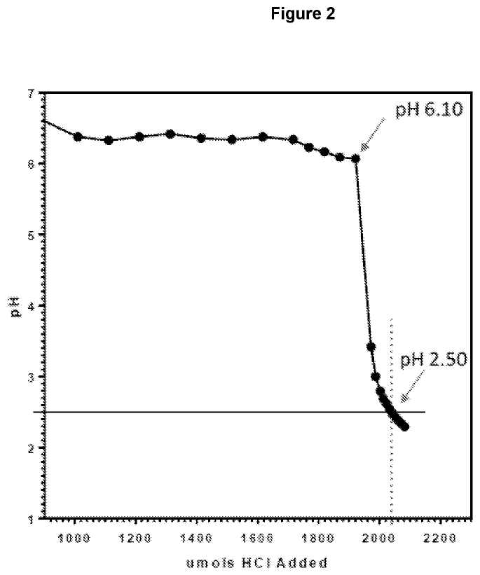

In the Experiment 2 titration, solid L-epinephrine free base powder was added to the water:alcohol oral delivery vehicle (** in Table 1), 2 N HCL was titrated in with stirring until a clear solution of pH 2.50 was achieved (see FIG. 2). An abrupt drop in pH occurred at pH 6.10 in which the turbidity also immediately clarified. Table 2 provides a summary of the pH values from this titration, and indicates the experimentally determined moles of HCl that were required to achieve pH 2.50 in this specific formulation

TABLE 2

| | | | Time (min) | |
|---|---|---|---|---|
| | | | to Discernible | |
| | | Appearance | Lip Blanch | Lip |
| | | of Beaker | (after applying | Blanch |
| Beaker Contents | pH | Contents | 1.0 ml to lips) | Response |
| 1. Orotopical delivery | 4.09 | Clear | | |
| vehicle** | | | | |
| 2. Added 20 mM L- | 6.72 | Turbid, | | |
| epinephrine free base | | chalky | | |
| (1000 umols) | | | | |
| 3. Added 535 ul of | 2.50 | Clear | 8 minutes | Clearly |
| 2N HCl in aliquots | | | | visible |
| (total = 1070 umol | | | | |
| HCl) | | | | |
| 4. Same as #3, +48 | 2.50 | Clear | | |
| hr at room temp. | | | | |

A 1.0 ml sample of the pH 2.50 20 mM L-epinephrine formulation was applied topically to a human test subject's lips to observe the time to induction of the blanch response in the oral mucosa tissue. The lip blanch results are summarized in Table 2 above.

At pH 2.50, the solution was clear and colourless, and it took 8 min to see a robust lip blanch response. At 48 hrs later, the drug solution was clear and colourless. We reasoned that in Experiment 1, though the drug formulation was clear to the eye at pH 4.95, there were insufficient moles of HCl to achieve complete dissolution and stabilization of the L-epinephrine free base, and this was detected as less dissolved, bioavailable, L-epinephrine drug molecules to induce lip blanch (hence the longer time to lip blanch and the blanch was weaker).

### Example 2

Experiments were conducted to determine the chemical stability of L-epinephrine when formulated as described in the “Experiment 2” section of Example 1. Previous work involved direct dissolution and formulation of commercially purchased “epinephrine HCl” crystals; they were dissolved directly into the orotopical formulation (“delivery/carrier vehicle”) described in Table 1 of Example 1; the final pH of this formulation is typically pH 5.05.

Following the HCl PH titration shown in Example 1 to the stable endpoint at pH 2.50, the applicant reasoned that the L-epinephrine formulated at pH 2.50 would be more chemically stable than the L-epinephrine at pH 5.05. At pH 5.05, the formulation is visibly “clear” to the eye, but it is literally at the centre of the precipitous pH drop that presumably accompanies ionization of the amine group on epinephrine in the pH 5-6 zone.

To quantitatively compare the chemical stabilities of L-epinephrine formulated either: 1. As we previously did by dissolution of L-epinephrine HCl crystals in oral “delivery vehicle” at a final pH of 5.05, or 2. As newly described in Example 1 by dissolution of L-epinephrine base powder in oral “delivery vehicle” and then adjusted pH to 2.50 with HCl, the applicant did the following:

1. L-epinephrine HCl crystals (MW: 219), to achieve 3.6 mg/ml final concentration, were dissolved by stirring in orotopical delivery vehicle consisting of: ethanol:glycerol:propylene glycol:water (6:6:8:80 vol:vol), 0.025% sucralose (wt:vol), 0.2% sodium metabisulfite, 0.1% citric acid. The measured pH was 5.05. 200 μl aliquots were delivered into 2.0 ml glass vials that were sealed with a Teflon-coated septum. (“epi-HCl vials”)

2. L-epinephrine free base (MW: 183), to achieve 20 mM (3.6 mg/ml) final concentration, was mixed by stirring in orotopical delivery vehicle consisting of: ethanol:glycerol:propylene glycol:water (6:6:8:80 vol:vol), 0.025% sucralose sweetener (wt:vol), 0.2% sodium metabisulfite, 0.1% citric acid. The measured pH was adjusted to 2.50 by the addition of 2 N HCl. 200 μl aliquots were delivered into 2.0 ml glass vials that were sealed with a Teflon-coated septum. (“epi-Base” vials)

3. Both the “epi-HCl” vials and the “epi-Base” vials were incubated under “stress conditions”, i.e., at 40° C. in the dark.

4. HPLC analysis of epinephrine peak area, both in each freshly prepared epi-HCl and epi-Base formulation, and in samples from stored vials over the next 10 days, was determined using a published HPLC protocol with a C18 reverse-phase column and elution with pH 2. 7% methanol solvent.

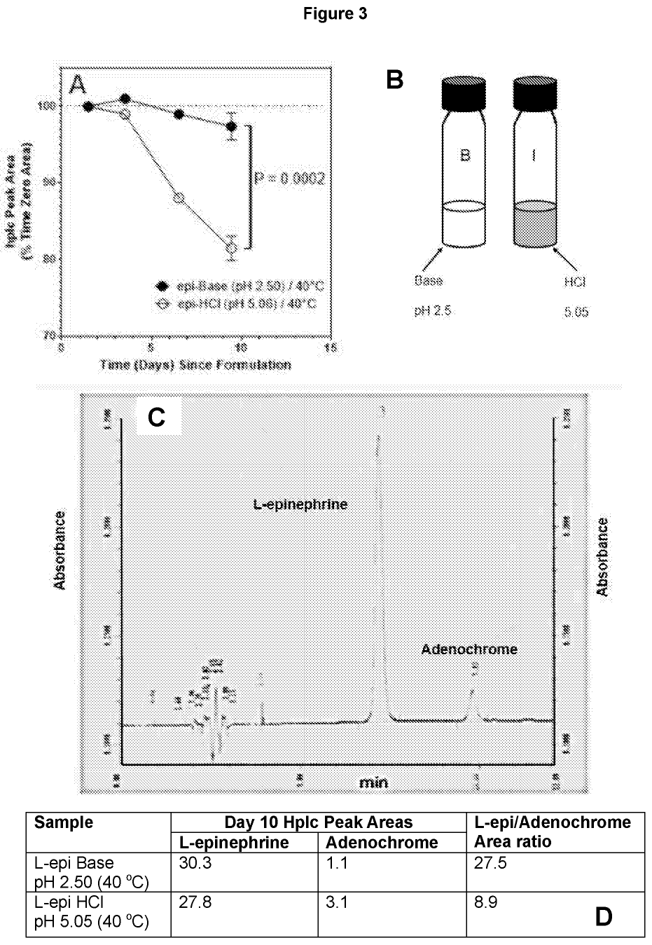

FIG. 3 shows the results of this comparative stability experiment.

Panel A shows a profound difference in epinephrine stability (P=0.0002), with no discernible degradation within the “epi-Base” formulation versus a 20% degradation within the “epi-HCl” formulation within the first 10 days of storage.

Panel B shows the rose-brown discoloration in the 10 day “epi-HCl” vials consistent with formation of the adrenochrome oxidative catabolite of epinephrine with no discernible discoloration in the “epi-Base” vial at 10 days.

Panel C shows the HPLC quantitation of both epinephrine and adrenochrome peaks in samples taken from a 10 day “epi-HCl” vial.

Panel D shows a substantial difference in the “L-epi/Adrenochrome Area Ratio” for samples taken from respective “epi-Base” and “epi-HCl” vials.

### Example 3

Experiments were conducted to determine the actual dose of L-epinephrine that would be retained on the topical surfaces of the human oropharyngeal cavity. In order to facilitate quantitation of the small drug molecule that is: i) delivered to the human oral cavity in 10-15 ml of delivery vehicle, ii) swished for 1-2.0 minutes, and iii) spit out after 2.0 minutes, a surrogate drug molecule, phenylephrine, was adopted as a substitute for L-epinephrine in this quantitative assay.

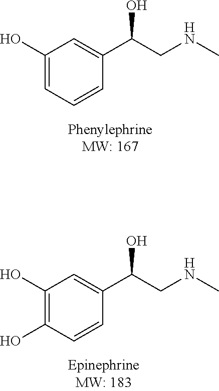

The applicant reasoned that phenylephrine would be an appropriate surrogate for L-epinephrine delivery because:
i) Both molecules are aromatic amines, and their molecular weights are only 8% different. Phenylephrine was originally invented to be an analog of epinephrine.
ii) HPLC chromatography analysis for phenylephrine was in place in our laboratory and the assay had been used in previous drug regulatory filings.

The applicant reasoned that the conditions of i) drug: vehicle administration volume and ii) drug: vehicle contact time with the oral mucosa that were optimum for phenylephrine dissolved in our orotopical delivery vehicle (i.e., Nitrogen gas-purged ethanol:glycerol:propylene glycol:water (6:6:8:80 vol:vol), with sweeter and antioxidants) would likewise be optimum for L-epinephrine delivery.

In these experiments, three human volunteer subjects were provided vials containing solution of 5 mM phenylephrine HCl dissolved in our orotopical delivery vehicle, and they were instructed to:
i) deliver the vial contents into their mouths;
ii) ii) swish the liquid in their mouths, pausing to gargle twice for five seconds; and
iii) at the specified time spit out the liquid into a glass vial.

The volume administered and the volume spit out by each test subject were recorded, and HPLC analysis of the phenylephrine concentration in the administered solution and the solutions spit out by each of the three volunteer subjects were determined. The results are summarised in Table 3.

TABLE 3

| | | | | Spit Out | % of | % of Orotopical | |
|---|---|---|---|---|---|---|---|
| | | Administered | | PhE-HCl | Administered | PhE-HCl | |
| | | PhE-HCl | Retrieved | mg/ml | Dose | Dose | mg PhE-HCl |
| | Administered | Dose | (Spit out) | (hplc) × ml | Spit Out | Delivered to | Actually |
| Subject # | Volume | (5 mM × 12.5 ml) A | Volume | B | B/A × 100 | Oral Surfaces | Delivered |
| 1 | 12.5 ml | 12.8 mg | 15.1 | 8.91, 9.51 (9.21) | 72 | 28 | 3.6 |
| 2 | 12.5 ml | 12.8 mg | 12.3 | 10.33, 10.95 | 83 | 17 | 2.2 |
| | | | | (10.59) | | | |
| 3 | 12.5 ml | 12.8 mg | 13.0 | 9.23, 9.62 (9.42) | 74 | 26 | 3.3 |

TABLE 4

| | | | μmols retained | Adult Human | Optimum L- |
|---|---|---|---|---|---|
| L-Epinephrine | Volume | | in oropharyngeal | Oropharyngeal | Epinephrine |
| Formulation | Administered | (A) μmols | cavity | Surface Area | Topical Dose |
| Concentration | (ml) | administered | (A × 0.24) | (cm²) | (μmols/cm²) |
| 10 mM | 12.5 ml | 125 | 30 | 188 | 0.160 |
| (1.8 mg/ml) | | | | | |
| 20 mM | 12.5 ml | 250 | 60 | 188 | 0.320 |
| (3.6 mg/ml) | | | | | |

Therefore, in three healthy adult human subjects, the mean percentage of the adrenergic vasoconstrictor actually retained on the orotopical surface of the subjects' mouths after the standard two-minute orotopical swish and gargle administration was: 24%

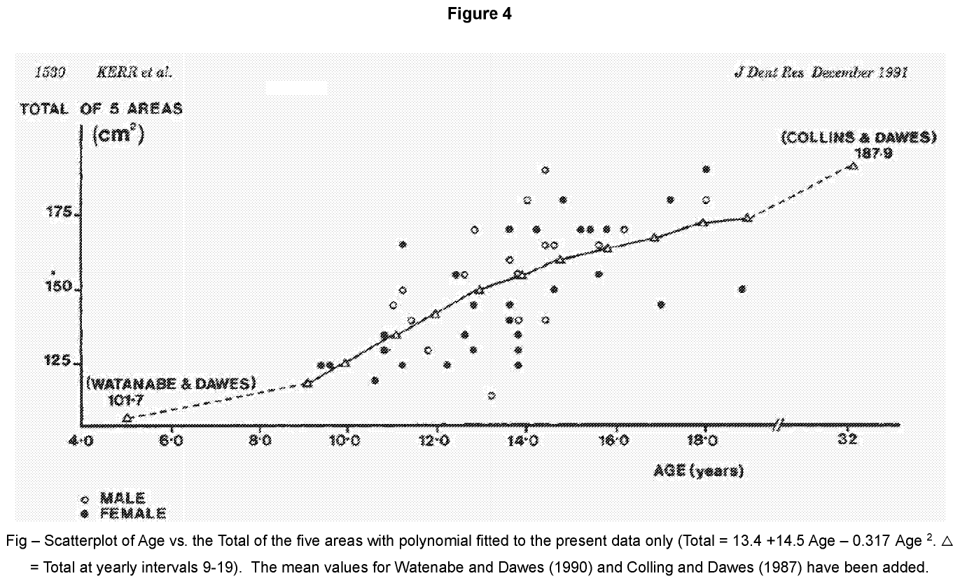

In the published data presented in FIG. 4 (Kerr et al.), adult humans are calculated to have an orotopical surface area of ˜188 cm².

These data shows that in the invention, when 1.8 mg/ml or 3.6 mg/ml L-epinephrine is administered to the mouths of adult humans in an empirically determined and optimised drug formulation and delivery protocol, which contains: i) an optimised delivery vehicle composition and ii) an optimised L-epinephrine chemical formulation, t about 20-30% of L-epinephrine dose from the orotopical solution is delivery to oral mucosa, as summarised in Table 4.

### Example 4

An experiment was done to determine whether the empirically optimised method of this invention, as described in the Examples and Claims of this patent application, for the formulation of L-epinephrine and its orotopical administration to human subjects, was effective in inducing submucosal blood vessels in a time and L-epinephrine dose-dependent manner that would enable its routine use in human subjects within the time constraints of a clinical setting. In the Example below, L-epinephrine was formulated and four healthy volunteers were treated as follows:

1. L-epinephrine powder (to achieve 0.9 mg/ml, 1.8 mg/ml, 3.6 mg/ml and 5.5 mg/ml final concentration) was added with stirring to an orotopical delivery vehicle which consisted of: Nitrogen gas-purged ethanol:glycerol:propylene glycol:water (6:6:8:80 vol:vol), 0.025% sucralose sweetener (wt:vol), and sodium metabisulfite and citric acid antioxidants.

2. While stirring, 2-5 N HCl was added to achieve a pH of 2.5.

3. 12.5 ml of the Step 2 L-epinephrine formulation was delivered oral topically to a human subject. The subject swished the 12.5 ml of formulation, gargling twice for 5 seconds each, for 2.0 minutes, and then spit out the formulation.

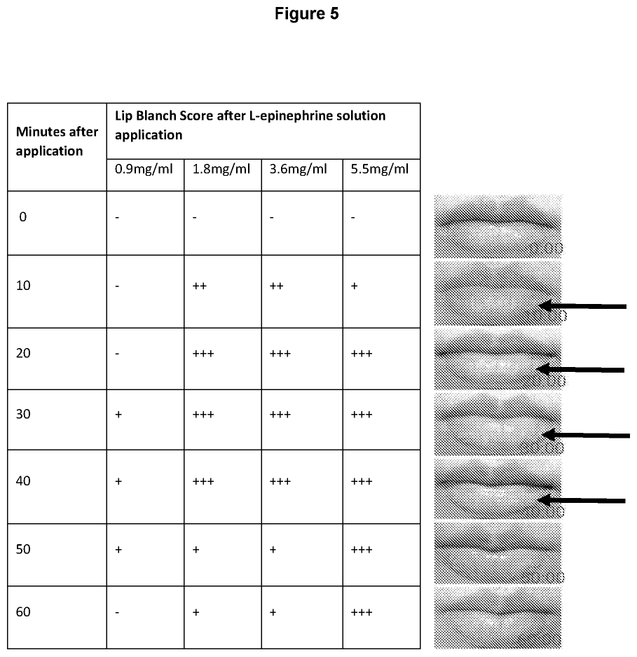

4. At 5 minutes after initiation of gargling, a photo of the subject's lips was taken which indicates a clear, robust blanch response (see arrow in FIG. 5) that resulted from delivery of L-epinephrine to the submucosal blood vessels.

A robust mucosal blanch of this nature, induced within 5-60 minutes of orotopical L-epinephrine administration to total of four healthy volunteers, is considered to be sufficient to induce a protective effect in a routine clinical radiotherapy and/or radiochemotherapy setting.

The results of this Example are illustrated in FIG. 5. The symbols in the Table, and their meanings are as follows: −no blanch respnse; +slight blanch response; ++moderate blanch response;

+++significant blanch response

### Example 6

Experiments were done to determine if L-epinephrine adrenergic vasoconstrictor was best suited to be used as the active pharmaceutical ingredient in formulations developed for delivery across mammalian squamous skin to achieve transient vasoconstriction, and the desired transient radioprotection and chemoprotection that accompany the vasoconstriction.

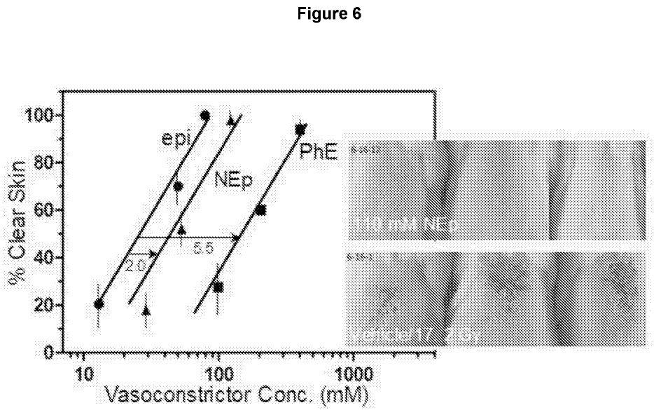

In a previous study, using a preclinical rat radiation dermatitis model (Fahl, Arch Dermatol Res 308:751-757, 2016), 75 μl of a formulation of each of the indicated vasoconstrictors in an ethanol:water (70:30) delivery vehicle was applied to a 4.5 cm² patch of shaved skin on a rat's back 12 min before the skin patch received a 17.2 Gy dose of radiation. Rats that received topical vehicle alone developed Grade 3 radiation dermatitis over >95% of the irradiated skin, and rats that received a prior topical application of vasoconstrictor showed a dose-dependent suppression of radiation dermatitis to background. FIG. 6 visually shows the suppression of radiation dermatitis and graphically shows the dose-dependency curves for each of the three adrenergic vasoconstrictors. The relative potencies of the topically applied vasoconstrictors was epinephrine: norepinephrine:phenylephrine at 1:0.5:0.2.

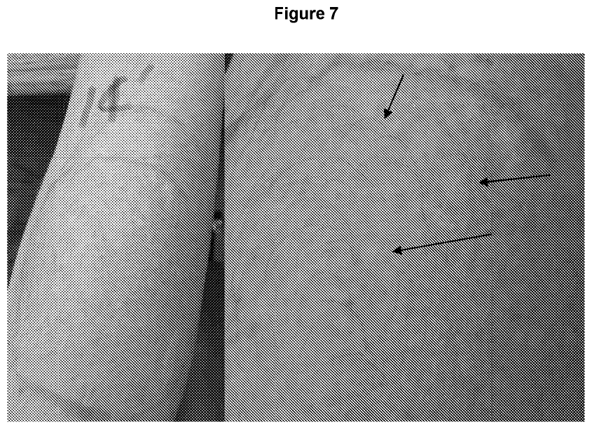

Following the animal study report, a skin blanching experiment on a healthy human volunteer's forearm was performed to indicate the vasocontriction effect of a formulation of L-epinephrine on squamous human skin. Dermal topical formulations containing water, 40-70% ethanol, antioxidants and karyolitic reagents were generated to dissolve L-epinephrine. As demonstrated in FIG. 7, an effective vasoconstriction skin blanching was achieved when a pharmaceutical formulation according to the invention containing 3 mg/ml L-epinephrine, water and 40% ethanol was topically applied to the skin. In particular, the skin blanching became significantly visible around the hair roots areas at 10-14 minutes after topical application. The blanching lasted about 40 minutes after the dermal topical application.

Both the animal study report and the skin blanching experiment allowed the empirical determination of the optimal alcohol:water topical delivery vehicles required for transmucosal and transcutaneous delivery, and allowed us to determine that L-epinephrine is a highly potent agent.

### Example 7

Experiments were conducted to determine conditions under which the L-epinephrine free base, which is the only chemical form of L-epinephrine available in the cGMP form acceptable for pharmaceutical formulation, could be solubilized in the water:alcohol-based delivery vehicle that had previously been determined to be best suited for delivery of catecholamine vasoconstrictors to mammalian squamous skin.

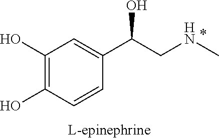

The need in the method claims of this patent is to deliver L-epinephrine to the subcutaneous blood vessels of human skin or scalp, in order to induce transient vasoconstriction of the blood vessels located 1 mm beneath the skin in order to provide transient protection against, such as: i) cancer radiotherapy to the skin or scalp, or ii) cancer chemotherapy delivered to scalp hair follicles, respectively.

It was reasoned that step-wise addition of L-epinephrine free base and titration with hydrochloric acid (HCl) would result in the formation of an HCl salt of the L-epinephrine amine group (* in Figure), and this would i) enable dissolution of the otherwise insoluble L-epinephrine base in the water:alcohol vehicle to achieve the required L-epinephrine concentration for mucosa, squamous skin and scalp delivery, and ii) with added antioxidants would provide stability to the dissolved L-epinephrine to enable the formulation and then holding in storage for a sufficiently long period.

In this Example, the volume of additional HCl is measured and a linear correlation between the HCl additional volume and the concentration of L-epinephrine is revealed.

| L-epi conc | pH without addition of | 2N HCl added to adjust |
|---|---|---|
| (mg/ml) | L-epi | to pH 2.33 (ml/l) |
| 0.00 | 3.69 | 0.63 |
| 0.92 | 4.32 | 5.36 |
| 5.50 | 7.10 | 11.88 |

A pharmaceutical formulation including ethanol, deoxidant and L-epinephrine drug substance has a pH above 6, hence excess amount of HCl is added into the formulation to obtain final pH lower than 3, preferably pH at about 2.3. The additional volume of HCl increases following a linear correlation to the concentration of L-epinephrine. The correlation can be described as the equation below:

$$Y = 5.6297 * X - 5.3 \; 044 , correlation ratio {R}^{2} = 0.9916$$

$$X \text{:} L - epinephrine concentration in mg / ml ;$$

$$Y \text{:} Additional volume of 2 N HCl in the final composition .$$

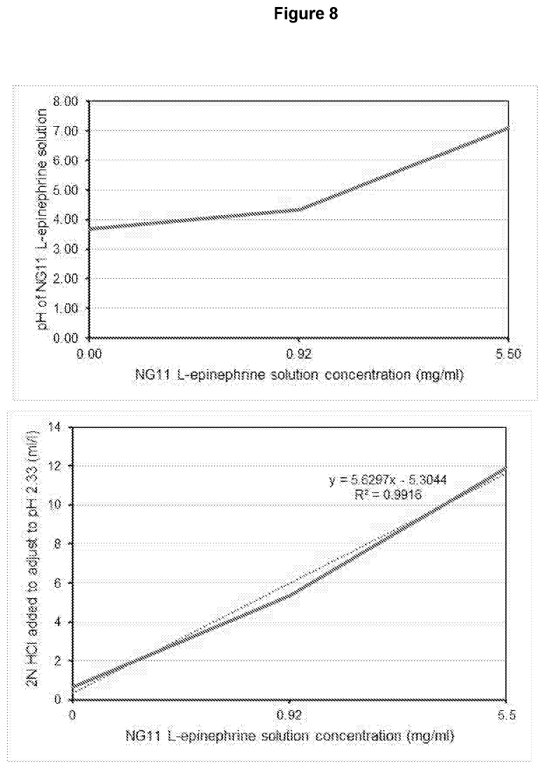

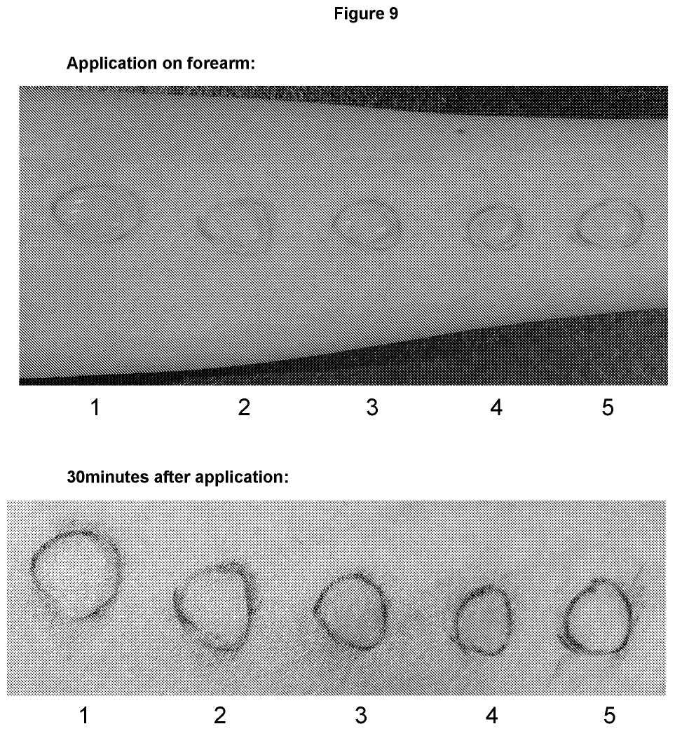

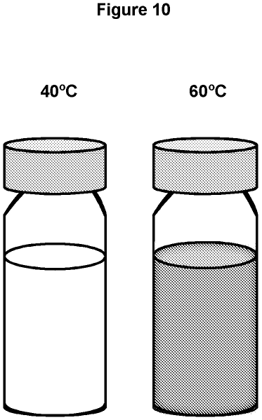

The correlation is calculated based on the measured collected during the production of the vehicle formulation without L-epinephrine, 0.92 mg/ml L-epinephrine and 5.5 mg/ml L-epinephrine formulation solutions (FIG. 8).

### Example 8

Experiments were conducted to determine what concentration of L-epinephrine, when formulated as described in Example 7 and applied topically to human squamous/scalp skin, would be needed to induce a durable skin blanch that would be expected to confer protection against chemotherapy and/or radiotherapy in cancer patients treated with either, chemotherapy of short (minutes) to long (hours) duration, or radiotherapy of a few seconds to a couple minutes duration per treatment. It was reasoned that by applying a known volume of L-epinephrine, formulated in the 70:30 (ethanol:water) vehicle detailed in Example 6-7, to a defined area of forearm skin on a human test subject, we could empirically determine the L-epinephrine concentration and topical application volume applied per unit of skin surface area that would confer a blanch response whose onset, degree, and duration would be suitable for specific radiotherapy or chemotherapy protection scenarios.

To determine these parameters, the following steps were carried out:
1. Formulated 3.4 mg/ml solutions of L-epinephrine in topical formulation 1) with ethanol, deoxidant and adjusted pH lower than 3, especially, pH 2.33; 2) with deoxidant and adjusted pH 2.33 but without ethanol; 3) with ethanol and adjusted pH 2.33 but without deoxidant; 4) with ethanol and deoxidant but with pH 5.5 without adjusted to lower than 3; 5) with ethanol, deoxidant and adjusted to pH 2.33, leave in daylight for 24 hours.
2. Oil pan draw five small circles on the forearm of a healthy volunteer including ˜1 cm² patches of skin to apply one droplet of each solution from the step 1 into one circle (see FIG. 9).
3. A clear skin blanching response can be seen in the circles with solution 1 and 5 at 30 minutes after applying the droplets of the five solutions.

The absence of blanching responses from the solution without ethanol, or without deoxidant, or without pH adjusted to lower than 3, further confirm the essential contributions to the bioavailability and stability of L-epinephrine from the combination of ethanol, deoxidant and pH lower than 3.

The known onset, degree and duration of the skin blanches can now be used to design and direct clinical trials in which topically applied L-epinephrine will be used to prevent radiation dermatitis or radiation-induced alopecia (short duration insult) as well as chemoradiotherapy-induced alopecia of a short duration insult. (such as Adriamycin-minutes).

### Example 9

The stability of pharmaceutical compositions comprising L-epinephrine with an oxygen concentration of less than 5% wt/vol and 20% wt/vol were tested and compared.

Stability of Pharmaceutical Compositions with 20% Wt/Vol Oxygen

The pharmaceutical composition comprising L-epinephrine at 5 mM (0.92 mg/ml) concentration was generated in chemical safety cabinet under normal air ventilation condition, and the oxygen concentration of the composition that was dispensed into the vials was subsequently replicated and measured to be around 20% wt/vol. The stability study samples were stored at the stability incubator chambers with various temperature and humidity conditions. At indicated time points, pulled samples were assessed on items such as Appearance, Identification, Colour of solution, Clarity of solutions, pH, Assay, Related Substances, Ethanol and Sodium Metabisulfite content for L-epinephrine solution.

There was a significant increase of total impurities more than 17% and the single impurity more than 8% under the 60° C. high temperature stress condition, the sample colour turned into light brown (FIG. 10) and the concentration of L-epinephrine decreased about 18%. Under 40° C. stress condition for 1 month, the total impurities of the sample solution reached to 3.8% and the single impurity reached nearly 1.7%. Notably, the concentration of antioxidant sodium metabisulfite under the condition of 60° C. and 40° C., especially under 60° C., showed a decreasing trend, may contribute to the large increase of impurities under the high temperature stress insults.

TABLE 10

| Test Item | | | | | | | | | | | |
|---|---|---|---|---|---|---|---|---|---|---|---|
| 5 mM | | | | | | | | | | | |
| (0.92 mg/ml) | | | Results-5 Days | | | Results-9 Days | | | Results-1 Month | | |
| Batch No. | Acceptance | | 60° C.- | 40° C.- | 15° C.- | 60° C.- | 40° C.- | 15° C.- | 60° C.- | 40° C.- | 15° C.- |
| 20190919 | Criteria | T0 | 5D | 5D | 5D | 9D | 9D | 9D | 1M | 1M | 1M |
| Description | Clear | CTP | CTP | CTP | CTP | CTP | CTP | CTP | CTP | CTP | CTP |
| | transparent | | | | | | | | | | |
| | solution (CTP) | | | | | | | | | | |
| Colour | Transparent (T) | T | T | T | T | T | T | T | light | T | T |
| | | | | | | | | | brown | | |
| Clarity | Clear (C) | C | C | C | C | C | C | C | C | C | C |
| pH | Report | 2.9 | 2.9 | 2.9 | 2.9 | 2.9 | 2.9 | 2.9 | 2.9 | 2.9 | 2.9 |
| Impurities | Not more than | 0.14% | 1.31 | 0.31 | 0.06 | 2.05 | 0.42 | 0.05 | 8.25% | 1.69% | 0.11% |
| | 0.5% of any | | | | | | | | (RRT0.15) | (RRT0.15) | (RRT0.15) |
| | single impurity | | | | | | | | | | |
| | Not more than | 0.18% | 3.6 | 0.93 | 0.24 | 6 | 1.2 | 0.24 | 17.65% | 3.84% | 0.94% |
| | 2.0% of Total | | | | | | | | | | |
| | impurities | | | | | | | | | | |
| Sodium | 0.4 mg/ml-2.2 | 0.635 | 0.4855 | 0.6019 | 0.5964 | 0.3929 | 0.5782 | 0.5841 | NA | NA | NA |
| metabisulfite | mg/ml | | | | | | | | | | |
| Assay | 92%-110% of | 100.1% | 96.35% | 99.08% | 99.76% | 93.94% | 98.75% | 99.76% | 82.34% | 96.16% | 99.04% |
| | labelled claim | | | | | | | | | | |

Stability of Pharmaceutical Compositions with 5% Wt/Vol Oxygen

In a separate experiment, the pharmaceutical compositions comprising L-epinephrine at 5 mM (0.92 mg/ml) and 30 mM (5.5 mg/ml) were produced in a deoxygenised closed isolator cabinet with oxygen monitoring to ensure the composition solutions were dispersed with final dissolved oxygen less than 5% wt/vol. The oxygen concentration in the solution in vials was measured using OxySense 325i meter. The stability of pharmaceutical compositions in accordance with the invention comprising a molecular oxygen concentration of less than 5% wt/vol was tested. The stability study samples were stored at various temperatures with other conditions same to the previous stability study. The stability results are shown in Table 11 and Table 12 below.

Batch 5 mM (0.92 mg/ml) and batch 30 mM (5.5 mg/ml) with dissolved oxygen concentration below 5% wt/vol showed little increase of single and total impurities at 2-8° C. storage condition and an acceptable level of total impurities at 25° C. storage condition for at least three months. In comparison to the composition samples with molecular oxygen concentration of about 20% wt/vol and stored at 40° C. for 1 month, the L-epinephrine composition samples with molecular oxygen concentration less than 5% wt/vol and stored at 40° C. showed significant improvement of stability on L-epinephrine concentration and impurity percentage, and the concentration of antioxidant sodium metabisulfite also remained stable.

TABLE 11

| Test Item | | | | | | | | | | |
|---|---|---|---|---|---|---|---|---|---|---|
| 5 mM | | | | | | | | | | |
| (0.92 mg/ml) | | | Results-1M | | | Results-2M | | Results-3M | | |
| Batch No. | Acceptance | | 40° C.- | 30° C.- | 25° C.- | 30° C.- | 25° C.- | 30° C.- | 25° C.- | 2-8° C.- |
| 20210517 | Criteria | T0 | 1M | 1M | 1M | 2M | 2M | 3M | 3M | 3M |
| Description | Clear | CTP | CTP | CTP | CTP | CTP | CTP | CTP | CTP | CTP |
| | transparent | | | | | | | | | |
| | solution (CTP) | | | | | | | | | |
| Colour | Transparent (T) | T | T | T | T | T | T | T | T | T |
| Clarity | Clear (C) | C | C | C | C | C | C | C | C | C |
| pH | Report | 2.9 | 2.9 | 2.9 | 2.9 | 2.9 | 2.8 | 2.8 | 2.8 | 2.9 |
| Impurities | Not more than | 0.04% | 1.08% | 0.29% | 0.17% | 0.65% | 0.30% | 0.78% | 0.37% | 0.04% |
| | 0.5% of any | | (RRT0.15) | (RRT0.15) | (RRT0.15) | (RRT0.15) | (RRT0.15) | (RRT0.15) | (RRT0.15) | (RRT0.15) |
| | single impurity | | | | | | | | | |
| | Not more than | 0.18% | 1.94% | 0.68% | 0.52% | 0.91% | 0.76% | 1.69% | 0.92% | 0.15% |
| | 2.0% of Total | | | | | | | | | |
| | impurities | | | | | | | | | |
| Sodium | 0.4 mg/ml-2.2 | 0.72 mg/ml | 0.59 mg/ml | 0.68 mg/ml | 0.54 mg/ml | 0.56 mg/ml | 0.58 mg/ml | 0.54 mg/ml | 0.55 mg/ml | 0.58 mg/ml |
| metabisulfite | mg/ml | | | | | | | | | |
| Assay | 92%-110% of | 97.4% | 96.4% | 96.8% | 97.4% | 96.7% | 97.1% | 93.3% | 94.3% | 95.2% |
| | labelled claim | | | | | | | | | |

TABLE 12

| Test Item | | | | | | | | | | |
|---|---|---|---|---|---|---|---|---|---|---|
| 30 mM | | | | | | | | | | |
| (5.5 mg/ml) | | | Results-1M | | | Results-2M | | Results-3M | | |
| Batch No. | Acceptance | | 40° C.- | 30° C.- | 25° C.- | 30° C.- | 25° C.- | 30° C.- | 25° C.- | 2-8° C.- |
| 20210514 | Criteria | T0 | 1M | 1M | 1M | 2M | 2M | 3M | 3M | 3M |
| Description | Clear | CTP | CTP | CTP | CTP | CTP | CTP | CTP | CTP | CTP |
| | transparent | | | | | | | | | |
| | solution (CTP) | | | | | | | | | |
| Colour | Transparent (T) | T | T | T | T | T | T | T | T | T |
| Clarity | Clear (C) | C | C | C | C | C | C | C | C | C |
| pH | Report | 3.2 | 2.9 | 2.9 | 2.9 | 2.8 | 2.8 | 2.8 | 2.8 | 2.9 |
| Impurities | Not more than | 0.04% | 1.59% | 0.55% | 0.35% | 1.04% | 0.57% | 1.41% | 0.82% | 0.13% |
| | 0.5% of any | | (RRT0.15) | (RRT0.15) | (RRT0.15) | (RRT0.15) | (RRT0.15) | (RRT0.15) | (RRT0.15) | (RRT0.15) |
| | single impurity | | | | | | | | | |
| | Not more than | 0.14% | 2.42% | 0.88% | 0.61% | 1.66% | 1.21% | 2.27% | 1.26% | 0.23% |
| | 2.0% of Total | | | | | | | | | |
| | impurities | | | | | | | | | |
| Ethanol | Report | 5.2% | 6.3% | 6.0% | 6.0% | 5.4% | 5.6% | 5.5% | 5.5% | 5.4% |
| Sodium | 0.4 mg/ml-2.2 | 0.69 mg/ml | 0.90 mg/ml | 0.99 mg/ml | 0.99 mg/ml | 0.75 mg/ml | 0.81 mg/ml | 0.61 mg/ml | 0.74 mg/ml | 0.83 mg/ml |
| metabisulfite | mg/ml | | | | | | | | | |
| Assay | 92%-110% of | 97.6% | 96.6% | 97.1% | 98.0% | 96.7% | 97.5% | 95.7% | 96.6% | 98.2% |
| | labelled claim | | | | | | | | | |

### Example 10

Pharmaceutical compositions of the invention were prepared according to the following protocol.

### 1. Disinfecting

Surfaces and equipment are preferably disinfected with 75% ethanol and dried them using clean compressed air. Since the pharmaceutical composition of the invention uses ethanol as a carrier, using at least 75% ethanol during this step can produce sufficient disinfection without introducing extra Process Related Impurities into the final product.

### 2. Washing and Drying of Vials

The vials are soaked and flushed with water, preferably purified water. The vials are then dried in a drying oven for at least 60 min at a temperature of 80-85° C. before being cooled to RT. Cooling of the vials is an important step to avoid ethanol excipient evaporation and API degradation in hot/warm vials.

### 3. Nitrogen Purging of Empty Compounding Vessel

Gas filters and related connecting pipes are attached to the vials for nitrogen filtration, and product filters and related connecting pipes are connected to the vials for product solution filtration. The nitrogen pressure is preferably equal or more than 0.2 MPa. The compounding vessel may be purged with filtered Nitrogen for at least 10 minutes until the oxygen concentration of the air in the vessel until the oxygen concentration is less than 5.0% wt/vol. The vessel is be flushed with nitrogen throughout the formulation process.

### 4. Compounding: Purified Water Charging

Preferably, approx. 70-80% of total weight of batch volume purified water is charged into the compounding vessel. The purified water is preferably stirred at a set speed of 200-800 rpm. While mixing, the nitrogen flushing in the compounding vessel is maintained and the solution should be kept bubbling at all times until the dissolved oxygen content of the purified water is no more than 5.0% wt/vol.

### 5. Compounding: Material Charging

The following components are then preferably dispensed to the vessel in the following order: citric acid, sucralose, propylene glycol, glycerol anhydrous, ethanol and sodium metabisulfite. Continue mixing at a set speed of 200-800 rpm for 5 minutes. Subsequently, L-epinephrine is added to the vessel. Adding the L-epinephrine last provides the best antioxidation effects and improved stability in the final product. The composition is preferably mixed at a speed of 200-800 rpm and circulated at a set speed of 14-16 rpm for at least 5 minutes.

The pH of the solution is adjusted to 2.0-2.5 using 2N hydrochloric acid solution, and the composition is stirred. The gross weight of the HCl solution (after pH adjusting) is recorded and the net weight of the hydrochloric acid is calculated.

After pH adjustment, purified water is added to the composition to reach the target net weight of the product. Stirring is continued at the speed of 200-800 rpm to obtain a homogenous solution.

### 6. Compounding: Circulation and Filtration

After charging purified water to full volume, the solution is circulated via a circulation system for 20 minutes as a minimum. The buffer tank is purged with nitrogen for 3-5 minutes and until the oxygen concentration is less than 5.0%. Depth filtration system commonly used for pharmaceutical GMP production is applied, and one step bag filters or two steps magnetic filters are used for the removal of solid impurities, undissolved powders, and particles to ensure a high-quality end product, and for reduction of bioburden to obtain sterile final products. At least one step of filtration with filter pore size of 0.22 μm is necessary to obtain sterile final solution. The filtrated solution is then charged to a buffer vessel for filling. Nitrogen flushing is maintained during the entire batch filling.

### 7. Filling Line Setting Up and Filling Weight Calculation

The filling system (pump, tubing and needles for product solution transferring and nitrogen blanketing pipe) is installed inside a closed RABS cabinet. The RABS cabinet is purged with filtered nitrogen for about 20 minutes and until the oxygen concentration of the air in the RABS is less than 5.0% wt/vol. Nitrogen flushing is maintained until the completion of filling.

### 8. Initial Vials Crimping Check

a. Sample 6 empty vials and insert a stopper into each of them using lip-off caps.
b. After crimping, the appearance of the crimped caps are checked. It should be tightly and crimped smoothly with no wrinkles.
C. Submit the bottles for an integrity test. Put these bottles into the sealing integrity tester and switch on the integrity testing machine. The testing pressure range is from −80.0˜-90.0 Kpa and the holding time is 180 s. Take them out from the tester, no water should be observed in the vials. Then the integrity test is passed.
d. If the results fail, adjust the crimping machine and submit another 6 bottles for integrity test.

The integrity test conditions are related to the vial size and filling volume. For the current 12.5 ml solution filling in 10 ml or 15 ml vial, this integrity test condition is adequate.

Repeat as necessary until the result passes and record.

### 9. Formal Filling and Closing

Transfer appropriate amount of empty vials and stoppers into the RABS. Flush the RABS with nitrogen and then purge the inner of all the vials briefly (3-5 s).

Sample 6 vials randomly and check the oxygen content. If the oxygen content is not more than 5.0%, then start the filling operation per the following procedures:
a. Fill the drug product solution into the bottle to the required weight range.
b. Immediately insert the stopper into the vial.
c. Then transfer all the stoppered vials from the RABS and crimp them with lip-off caps using capping machine as soon as possible.

Repeat the above filling and stoppering campaign inside the RABS and crimping operations until the completion of the entire batch. For each filling and stoppering campaign inside the RABS, randomly sample 6 pieces of the crimped vials for IPC test. Thereof 3 vials for weight check and the remaining 3 vials for oxygen content test.

At the start, middle and end of the filling and closing, perform seal integrity check accordingly (using 3 empty bottles per time) and record integrity testing parameters.

Collect all the waste product solution and record the weight. The IPC samples, unused product solution will be discarded.

### 10. Reviewing and Recording

Check and record the temperature, relative humidity and pressure differential after completion of compounding, filling and closing operations.

### EMBODIMENTS

It is envisaged that the invention may explicitly exclude any one of the following embodiments.

1. A pharmaceutical L-epinephrine composition based on an ethanol:glycerol : propylene glycol:water (6:6:8:80 vol:vol) vehicle having a molecular oxygen concentration of less than 5% (wt:vol) comprising between 5 mM and 30 mM L-epinephrine, and an 0.9 to 1.3 molar excess of HCl, relative to the L-epinephrine, wherein the composition also contains
0.01 to 0.5% (wt:vol) of one or more antioxidants;
0.01 to 5% (wt:vol) of one or more sweeteners;
0.05 to 2% (wt:vol) of one or more flavoring agents.

2. The pharmaceutical composition according to embodiment 1, wherein the vehicle has a molecular oxygen concentration of less than 2-5% (wt:vol).

3. The pharmaceutical composition according to embodiment 1, wherein the L-epinephrine is present at 5 mM, 10 mM, 15 mM, 20 mM or 30 mM.

4. The pharmaceutical composition according to embodiment 1, wherein the antioxidant is metabisulfite or citric acid, or a combination thereof.

5. The pharmaceutical composition according to embodiment 4 containing as the antioxidants 0.2% (wt:vol) metabisulfite and 0.1% (wt:vol) citric acid.

6. The pharmaceutical composition according to embodiment 1, wherein the sweetener is sucralose.

7. The pharmaceutical composition according to embodiment 6 containing as the sucralose sweetener 0.025% (wt:vol) sucralose.

8. The pharmaceutical composition according to embodiment 1 wherein the flavouring agent is Cherry Flavor.

9. The pharmaceutical composition according to embodiment 1 containing as the flavouring agent 0.28% (wt:vol) Cherry Flavor.

10. Use of a pharmaceutical composition according to any previous embodiment for the prevention of radiation-induced and/or chemoradiotherapy-induced oral mucositis in oncology patients undergoing oncology therapies including radiation exposure to oral mucosa.

11. A method in which the pharmaceutical composition of embodiment 1 is administered to the oral and oropharyngeal cavity of a patient undergoing oncology radiotherapy and/or chemoradiotherapy in which 10.0-15.0 ml of pharmaceutical composition is administered, and the formulation is i) maintained and swished within the oral and oropharyngeal cavity for 1.5-2.5 minutes, ii) excess formulation is then spit out, and iii) the administered L-epinephrine achieves a final orotopical dose range of 0.1-0.5 μmols L-epinephrine/cm² of oral surface.

12. A method in which the pharmaceutical composition of embodiment 1 is administered to the oral and oropharyngeal cavity of a patient undergoing oncology radiotherapy and/or chemoradiotherapy within 40-50 minutes before each fractional radiation exposure to oral mucosa.

13. A pharmaceutical L-epinephrine composition based on an ethanol:water (70:30 vol:vol) vehicle having a molecular oxygen concentration of less than 0.5% (wt:vol) comprising between 50 mM and 200 mM L-epinephrine, and an 0.8 to 1.2 molar excess of HCl, relative to the L-epinephrine, which composition also contains 0.01 to 0.5% (wt:vol) of one or more antioxidants.

14. A method in which the pharmaceutical composition of embodiment 13 is administered to squamous skin and/or the scalp of a patient undergoing cancer radiotherapy and/or chemotherapy in which 20 ml to 30 ml of pharmaceutical composition is topically administered, and the administered L-epinephrine achieves a final topical dose of 1.0-4.0 μmol L-epinephrine/cm² of squamous skin and/or scalp surface.

### REFERENCE

Alvariño-Martín C, Sarrión-Pérez MG. Prevention and treatment of oral mucositis in patients receiving chemotherapy. J Clin Exp Dent. 2014 Feb. 1; 6 (1): e74-80. doi: 10.4317/jced.51313. eCollection 2014 February.
Chaveli-López B, Bagan-Sebastian J V. Treatment of oral mucositis due to chemotherapy. J Clin Exp Dent. 2016 Apr. 1; 8 (2): e201-9. doi: 10.4317/jced.52917. eCollection 2016 April.
Cleary J F, Anderson B M, Eickhoff J C, Khuntia D, Fahl W E. Significant suppression of radiation dermatitis in breast cancer patients using a topically applied adrenergic vasoconstrictor. Radiat Oncol. 2017 Dec. 22; 12 (1): 201.
De Sanctis V, Bossi P, Sanguineti G, Trippa F, Ferrari D, Bacigalupo A, Ripamonti C I, Buglione M, Pergolizzi S, Langendjik J A, Murphy B, Raber-Durlacher J, Russi E G, Lalla R V. Mucositis in head and neck cancer patients treated with radiotherapy and systemic therapies: Literature review and consensus statements. Crit Rev Oncol Hematol. 2016 April; 100:147-66.Doi: 10.1016/j.critrevonc.2016.01.010. Epub 2016 Feb. 1.
Fahl W E., Complete prevention of radiation-induced dermatitis using topical adrenergic vasoconstrictors. Arch Dermatol Res. 2016 December; 308 (10): 751-757.
Graul-Conroy A, Hicks E J, Fahl W E., Equivalent chemotherapy efficacy against leukemia in mice treated with topical vasoconstrictors to prevent cancer therapy side effects. Int J Cancer. 2016 Jun. 15; 138 (12): 3011-9. doi: 10.1002/ijc.30037. Graul-Conroy A, Hoover-Regan M, DeSantes K B, Sondel P M, Callander N S, Longo W L, Fahl W E. Reduction in oral mucositis severity using a topical vasoconstrictor: A case report of three bone marrow transplant patients. Integr Cancer Sci Ther. 2018 December; 5 (6): 10.15761/ICST.1000293. doi: 10.15761/ICST.1000293. Epub 2018 Nov. 29. PMID: 31832233; PMCID: PMC6907163.
Maria O M, Eliopoulos N, Muanza.T, Radiation-Induced Oral Mucositis. Front Oncol. 2017; 7:89. 2017.
Soref C M, Fahl W E., A new topical vasoconstrictor-based strategy for prevention of oral mucositis. Oral Surg Oral Med Oral Pathol Oral Radiol. 2014 April; 117 (4): 454-61.
Soref C M, Fahl W E., A new strategy to prevent chemotherapy and radiotherapy-induced alopecia using topically applied vasoconstrictor. Int J Cancer. 2015 Jan. 1; 136 (1): 195-203.

1. A pharmaceutical composition comprising:
0.9 mg/ml to 15 mg/ml L-epinephrine or salts thereof; and a carrier vehicle comprising ethanol and water;
wherein the pharmaceutical composition has a molecular oxygen concentration of less than 5% wt/vol; and
wherein the pharmaceutical composition has a pH of less than 3.

2. The pharmaceutical composition according to claim 1, wherein the composition comprises hydrochloric acid.

3. The pharmaceutical composition according to claim 1, wherein the carrier vehicle further comprises glycerol and/or propylene glycol.

4. The pharmaceutical composition according to claim 1, wherein the L-epinephrine or salts thereof are present in an amount of 0.9 mg/ml to 11 mg/ml.

5. The pharmaceutical composition according to claim 1, wherein the pharmaceutical composition has a molecular oxygen concentration of less than 4.5% wt/vol.

6. The pharmaceutical composition according to claim 1, further comprising D-epinephrine, DL-epinephrine and/or salts thereof.

7. The pharmaceutical composition according to claim 1, wherein the composition further comprises one or more antioxidants.

8. The pharmaceutical composition according to claim 1, wherein the composition further comprises one or more penetration enhancers.

9. The pharmaceutical composition according to claim 1, wherein the composition further comprises one or more keratolytic ingredients.

10. The pharmaceutical composition according to claim 1, wherein the composition further comprises one or more sweeteners; and/or
the composition further comprises one or more flavouring agents.

11. The pharmaceutical composition according to claim 1, wherein the composition is formulated as a mouth rinse, gel, balm, oil, paste or aerosol.

12. A method for preventing chemotherapy-induced and/or radiotherapy induced oral mucositis in a patient and/or changes to speech and voice in a patient; the method comprising administering an orotopical formulation of the pharmaceutical composition of claim 1 to an oral surface or oropharyngeal cavity surface of the patient with or without an applicator device.

13. The method according to claim 12, wherein the composition is in a dose suitable to provide from 0.03 milligram-3 milligram L-epinephrine/cm² on the treated oral mucosa surface or oropharyngeal cavity surface of the patient.

14. The method according to claim 12, wherein the composition is administered to the oral mucosa surface or oropharyngeal cavity surface of a patient undergoing radiotherapy and/or chemoradiotherapy no more than 50 minutes prior to a first and each subsequent fractional radiation exposure to the treated oral or oropharyngeal cavity surface.

15. A pharmaceutical composition comprising:
0.92 mg/ml to 36.6 mg/ml L-epinephrine or salts thereof;
and a carrier vehicle comprising ethanol and water;
wherein the pharmaceutical composition has a molecular oxygen concentration of less than 5% wt/vol; and
wherein the composition has a pH of less than 3.

16. The pharmaceutical composition according to claim 15, wherein the composition comprises hydrochloric acid.

17. The pharmaceutical composition according to claim 15, wherein the carrier vehicle comprises:
from 30% to 80% vol/vol ethanol; and
from 20% to 70% vol/vol water.

18. The pharmaceutical composition according to claim 15, further comprising one or more antioxidants.

19. The pharmaceutical composition according to claim 15, wherein the composition is formulated as a gel, shampoo, balm, oil, paste or cream.

20. A method for preventing radiotherapy induced conditions selected from the group comprising dermatitis, alopecia, acute skin inflammation, rash, ulceration, mucosa ulceration, decolouration, pigmentation, scaring and chronic fibrosis in a patient; the method comprising administering a topical formulation of the composition of claim 15 to the squamous skin and/or the scalp of a patient.

21. The method according to claim 20, wherein the composition is in a dose suitable to provide from 0.03 mg to 3 mg L-epinephrine/cm² on the treated squamous skin surface or scalp surface and the hair follicles of a patient.

22. The method according to claim 20, wherein the composition is administered to the squamous skin surface or scalp surface of a patient undergoing radiotherapy and/or chemoradiotherapy between to 50 minutes prior to a first and each subsequent fractional radiation exposure to the treated squamous skin or scalp surface.

23. A method of forming a pharmaceutical composition comprising the steps of:
a) adding water to a vessel;
b) purging the water in the vessel with nitrogen gas until the dissolved molecular oxygen content reaches 5% wt/vol or less;
c) adding ethanol and at least one antioxidant to the vessel to form a composition;
d) adding at least one alpha-adrenergic vasoconstrictor to the composition; and
e) adjusting the pH of the composition to less than 3;
wherein the composition has a molecular oxygen concentration of less than 5% wt/vol.

24. The method according to claim 23, wherein the antioxidant is selected from the group comprising citric acid, vitamin C, vitamin E, L-cysteamine, a bisulfite salt, or combinations thereof.

25. The method according to claim 23, wherein the alpha-adrenergic vasoconstrictor is selected from L-epinephrine, D-epinephrine, norepinephrine, phenylephrine or mixtures thereof.

26. The method according to claim 23, wherein at least one stabiliser is added to the composition in step c).

27. The method according to claim 23, wherein at least one flavouring agent is added to the composition in step c).

28. The method according to claim 23, wherein in step e) the pH of the composition is adjusted with hydrogen chloride.

29. The method according to claim 23, wherein the method further comprises the step of:
f) filtering the composition with filter pore sizes of 0.3-0.5 μm and then 0.18-0.28 μm.

30. The method according to claim 23, wherein the method further comprises the step of purging the air in the vessel with nitrogen prior to step a) until the oxygen concentration of the air in the vessel is less than 5% wt/vol.

31. The method according to claim 23, wherein the method further comprises the step of vial filling in a closed nitrogen environment to control the oxygen concentration dissolved in the final solution and vial headspace aerosol both below 5%.

32. The pharmaceutical composition according to claim 3, wherein the carrier vehicle comprises:
from 2% to 10% vol/vol ethanol;
from 2% to 10% vol/vol glycerol;
from 4% to 12% vol/vol propylene glycol; and
from 70% to 90% vol/vol water.

33. The pharmaceutical composition according to claim 7, wherein the one or more antioxidants is present in an amount of from 0.01 to 0.5% wt/vol.

34. The pharmaceutical composition according to claim 10, wherein the one or more sweeteners is present in an amount of from 0.01 to 5% wt/vol; and
the one or more flavouring agents is present in an amount of from 0.05 to 2% wt/vol.

35. The pharmaceutical composition according to claim 18, wherein the one or more antioxidants is present in an amount of from 0.01 to 0.5% wt/vol.

36. The method according to claim 26, wherein the stabiliser is glycerol, propylene glycol, or mixtures thereof.
